(19) United States
(12) Patent Application Publication
(10) Pub. No.: US 2009/0036656 A1
(43) Pub. Date: Feb. 5, 2009

(54) Method for preparing a biomaterial

(75) Inventors: Horng-Ji LAI, Taipei (TW); Chien-Cheng LIN, Taipei City (TW); Shang-Ming LIN, Fongyuan City (TW)

Correspondence Address: BIRCH STEWART KOLASCH & BIRCH, PO BOX 747, FALLS CHURCH, VA, 22040-0747, US

(73) Assignee: BODY ORGAN BIOMEDICAL CORP., Taipei (TW)

(21) Appl. No.: 12/081,015

(22) Filed: Apr. 9, 2008

Related U.S. Application Data
(63) Continuation-in-part of application No. 11/882,328, filed on Jul. 31, 2007.

(51) Int. Cl. C07K 1/14; C07K 14/78; A61K 38/17

(52) U.S. Cl. 530/427; 530/857

(57) ABSTRACT

A method of preparing a biomaterial comprises steps of acellularizing the fish scale, decalcifying the fish scale, and cleaning the fish scale and then extruding the fish scales.

### BACKGROUND OF THE INVENTION

The present invention relates to a method for preparing a biomaterial, and particularly to a biomaterial prepared from fish scales for use in tissue repair and implantation.

Tissue engineering that involves the incorporation of a biomaterial with biologics and/or pharmaceutics and upon implantation in a patient will stimulate angiogenesis, tissue integration, and/or tissue remodeling. The biomaterial is a synthetic and biocompatible material that is used to construct artificial organs, rehabilitation devices, or prostheses and replace natural body tissues.

For over decades, collagen fiber, hydroxyapatite (HAP) and tri-calcium phosphate (TCP) are some biomaterials with great biocompatibility and safety to be used in human tissue implant. However, these biomaterials have disadvantages such as low mechanical strength, risk of chemical residue in cross linking, terrestrial animal transmitted disease.

Therefore, it is desirable to develop a biomaterial having a high mechanical strength, low possibility of contracting with the terrestrial contagious disease and is applicable to tissue repairs or implants.

### BRIEF SUMMARY OF THE INVENTION

It is an aspect of the invention to provide a biomaterial prepared from fish scales by a process which includes steps of acellularizing the fish scale, decalcifying the fish scale, and cleaning the fish scale and then extruding the fish scales.

The process further comprises a step of dehydrating the fish scales until the fish scales contain less than about 50% of water and a step of soaking the scale. In an embodiment of the invention, the fish scales contain less than about 25% of water.

It is another aspect of the invention to provide a biomaterial prepared from fish scales by a process, which comprises subjecting the fish scales to a heat treatment at a temperature of less than about 200° C.

It is yet another aspect of the invention to provide a use of the biomaterial prepared by the process described above for repairing tissues.

It is yet a further aspect of the invention to provide a use of the biomaterial prepared by the process described above for tissue implantation.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A to 1F are flow charts illustrating six processes for preparing a biomaterial with simple contents.

### DETAILED DESCRIPTION OF THE INVENTION

Reference will now be made in detail to the present embodiments of the invention, examples of which are illustrated in the accompanying drawings.

The process for preparing a biomaterial from scales 10 in the present invention comprises acellularizing the fish scale 10 (S11), decalcifying the fish scale 10 (S12), and cleaning the fish scale 10 (S13) and then extruding the fish scales 10 (S16).

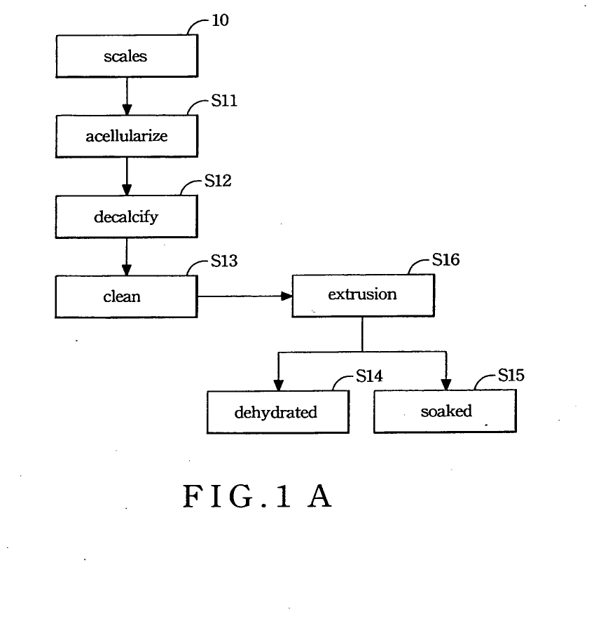

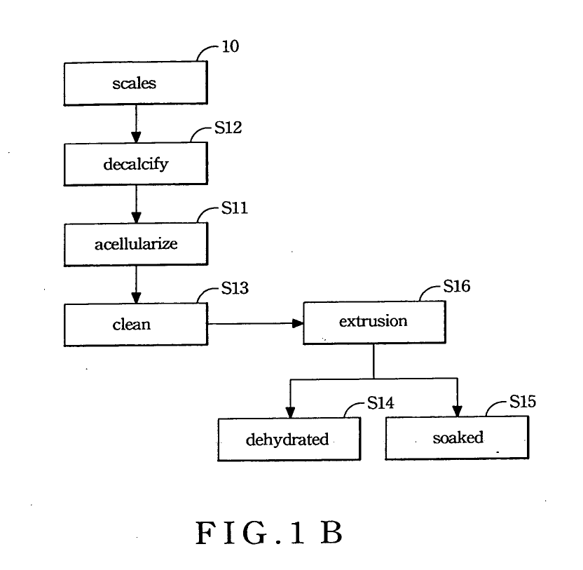

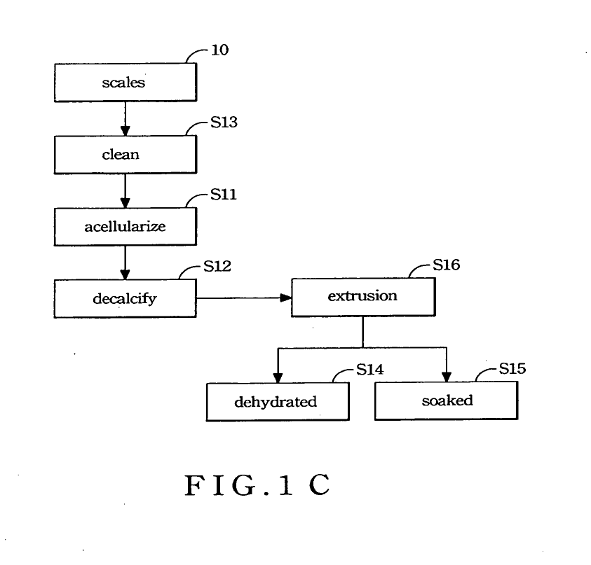

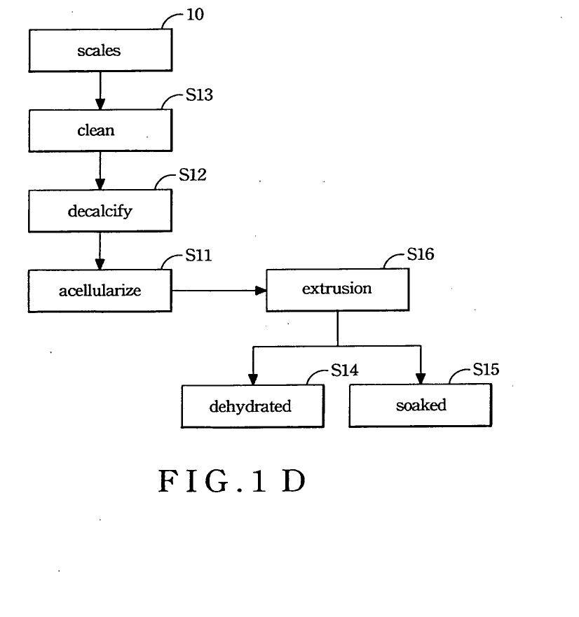

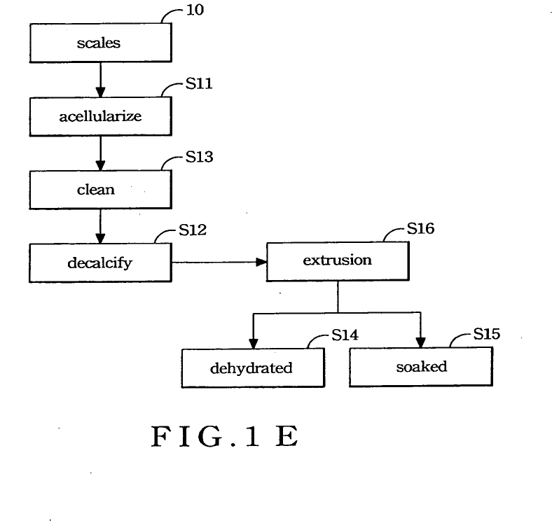

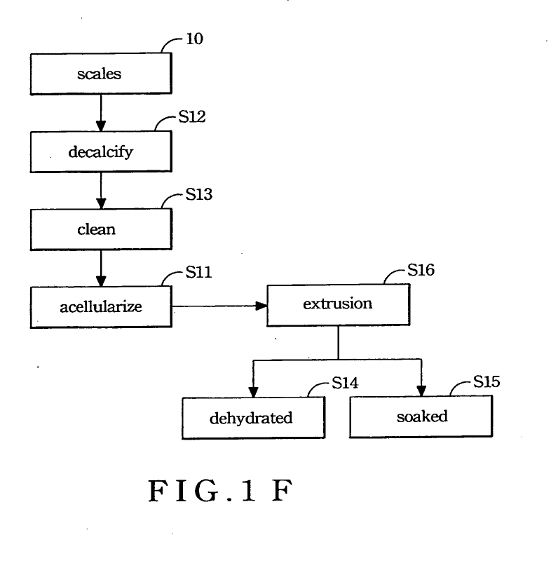

Referring to FIG. 1A to 1F, the method for preparing the biomaterial from the fish scale 10 of the present invention wherein, the steps of acellularizing the fish scale 10 (S11), decalcifying the fish scale 10 (S12), and cleaning the fish scale 10 (S13) does not limit the acting order. The three steps can exchange the order mutually. It will have six kinds of embodiment as FIG. 1A to 1F, and all of them can arrive the purpose of the present invention.

In accordance with some examples of the invention, the fish scales 10 may be freshly provided in a chilled or frozen manner. In a specific example of the invention, the fish scales 10 each having an average size less than about 20 cm in diameter may be selected for preparing the biomaterial. Using non-ionic solution to acellularize (S11) the fish scales 10 avoids residual cells affecting the biocompatibility. Because taking off the calcium content in the fish scales 10 can increase transparency, using acid solution to decalcify (S12) the fish scales 10 will increase transparency of the biomaterial. The purpose of cleaning (S13) is getting rid of the impurities of the fish scales 10. The fish scales 10 may be cleaned by washing (S13) with other cleaning agents including but not limited to surfactant, detergent, warm water and polar solvent such as ethanol. However, the present invention is not limited to any particular cleaning step. For example, the fish scales 10 are cleaned enough to pass Limulus Amebocyte Lysate (LAL) test which is an assay for detection and quantitation of bacterial endotoxin. Preferably, the cleaned fish scales would have a LAL test score less than about 1000 Eu/ml.

No matter what kind of the order of the three steps (S11), (S12), (S13) proceed, the step of extruding the scale should proceed after the three steps (S11), (S12), (S13) totally complete.

The method for preparing a biomaterial of the present invention further comprises a step of dehydrating the scale (S14) and a step of soaking the scale (S15). The fish scales 10 may be dehydrated (S14) and soaked the scale (S15) (but not limited) after the three steps (S11), (S12), (S13) totally complete, too. In a preferred embodiment, the step of dehydrating the scale (S14) and soaking the scale (S15) proceed after the step of extruding the scale (S16). The fish scales 10 may be dehydrated by air spraying, oven, freeze drying or any other conventional dehydration methods available so far. Also, the fish scale may be dehydrated by soaking the fish scales in the ethanol or other polar organic solvent. The fish scales are dehydrated until their water content is less than about 50%, preferably less than about 25%. Furthermore, the fish scales are soaked in isotonic solution of target tissue, such as 0.9% normal saline.

These products may be further processed, for example, by dehydrated (S14), soaked (S15), extrusion (S16), fully or partially drying and sterilizing to yield sterilized biomaterials. In a preferred embodiment, these steps may be performed with or without heating. These products may be used in combination with a variety of connective tissue repair compositions, or in combination with other active or inactive ingredients.

Referring to FIG. 1A to 1F, the detailed method for preparing the biomaterial from the fish scale of the present invention wherein, the steps of dehydrating the fish scale 10 (S14), and extruding the fish scale 10 (S16) does not limit the acting order, but they should proceed after the three steps (S11), (S12), (S13), and (S16).

In one other example, the dehydrated fish scales (S14) may be subjected to a heat treatment, such as a extrusion (S16) process performed at a temperature of less than about 200° C., preferably, about from 110° C. to 200° C. The intact fish scales or different types of biomaterials derived from fish scales, previously cold pressed with a pressure of more than 100 g in 2.5 cm³, preferably, more than 1 kg in 2.5 cm³, are submitted to hot pressing performed at a temperature of less than about 200° C. in a desired mold. The cross linking of the biomaterials can be achieved physically by heating or chemically by adding with a cross linker at an optimal concentration before extrusion is performed. The cross linker is reactive with the amines group or other reactive group in the biomaterials. The dehydrated fish scales (S14) may be subjected to the extrusion (S16) with or without one or more cross linking ingredients. The heat treatment in the present invention is not limited to the extrusion (S16) described above. One skilled in the art may also adopt other heat treatments such as thermal extrusion of any type, thermal pressing and molding steps to produce the biomaterial.

The biomaterial of the invention contains tissue repair factors and may be manufactured into a tissue repair material for repairing a variety of tissue damages and tissue defect sites. For example, the biomaterial of the invention may be prepared for soft tissue defect sites or be a coating material coated on surgical grafts or implants to be implanted at, into, onto defect sites, such as cornea tissue, tympanum tissue or other soft tissue defect sites. Accordingly, the invention is also applicable to connective tissue surgical implant with the tissue repair material derived from fish scales, whereby the surgical implant is implanted at a connective tissue defect site.

Summarizing from the above, the invention relates to a biomaterial prepared from the fish scales for use in a variety of tissue repairs and implantations. These fish scale derived products may contain tissue repair factors and may be further processed to produce a variety of formulations and consistencies.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

I/We claim:

1. A method for preparing a biomaterial from scales, comprising the step of:
acellularizing a scale and decalcifying the scale.

2. The method according to claim 1, further comprising a step of cleaning the scale.

3. The method according to claim 2, further comprising a step of dehydrating the scale after cleaning the scale.

4. The method according to claim 3, wherein the step of dehydrating the scale comprising dehydrating the scale until the scale containing less than about 50% of water.

5. The method according to claim 1, further comprising a step of extruding the scale.

6. The method according to claim 5, wherein the extrusion is performed at a temperature of less than about 200° C.

7. The method according to claim 5, further comprising a step of soaking the scale in water.

8. The method according to claim 1, wherein the average diameter of the scale is less than 20 cm.

9. The method according to claim 1, wherein the scale is a fish scale.

10. The method according to claim 1, further comprising a step of acetifying the scale when decalcifying the scale.
